(19) United States
(12) Patent Application Publication
(10) Pub. No.: US 2007/0191912 A1
(43) Pub. Date: Aug. 16, 2007

(54) Interactive electrical stimulator device and server-based support system

(75) Inventors: Wallace Ray Fischer, Amesville, OH (US); Raymond Charles Fischer, Amesville, OH (US); Alan Wade, Irvine, CA (US); Michelle Pinard, Irvine, CA (US); Mindy Black, Fountain Valley, CA (US)

Correspondence Address: MCDERMOTT WILL & EMERY LLP, 18191 VON KARMAN AVE., SUITE 500, IRVINE, CA, 92612-7108, US

(73) Assignee: Vision Quest Industries, Inc., Irvine, CA (US)

(21) Appl. No.: 11/704,601

(22) Filed: Feb. 9, 2007

Related U.S. Application Data
(60) Provisional application No. 60/772,093, filed on Feb. 10, 2006.

(51) Int. Cl. A61N 1/18

(52) U.S. Cl. 607/60

(57) ABSTRACT

A portable electro-therapy device includes a microprocessor configured to generate and control electrical stimulation output signals, an electrode jack, a plurality of electrodes operatively connected to the electrode jack and configured to transmit the electrical stimulation output signals to a patient, a modem communication port, and a modem configured to provide communication between the portable electro-therapy device and a server-based support system. A server-based support system includes an application server configured to provide an interface to the server-based support system to authorized users, a resources server configured to securely store patient data, and a modem bank configured to provide communication between the portable electro-therapy device and the server-based support system.

### CROSS-REFERENCE TO RELATED APPLICATION

The present application claims the benefit of priority under 35 U.S.C. §119 from U.S. Provisional Patent Application Ser. No. 60/772,093 entitled “INTERACTIVE ELECTRICAL STIMULATOR DEVICE AND SERVER-BASED SUPPORT SYSTEM,” filed on Feb. 10, 2006, the disclosure of which is hereby incorporated by reference in its entirety for all purposes. This application is also related to commonly-owned U.S. Pat. No. 6,564,103 B2, entitled “ELECTRICAL STIMULATOR AND METHOD OF USE,” issued May 13, 2003, which is incorporated herein by reference in its entirety for all purposes.

### STATEMENT REGARDING FEDERALLY SPONSORED RESEARCH OR DEVELOPMENT

Not applicable.

### FIELD OF THE INVENTION

The present invention generally relates to a portable multi-mode electrical electro-therapy for performing electrotherapy on a patient, and a server-based system which communicates with the portable electro-therapy for loading therapy protocols and for monitoring usage and therapy progress.

### BACKGROUND OF THE INVENTION

Portable electro-therapy or stimulator devices can provide one or more modes of therapy for a patient. For example, the above-referenced U.S. Pat. No. 6,564,103 B2 describes a portable electro-therapy device which is capable of providing at least three modes of stimulation therapy: high voltage pulsed current (“HVPC”), Neuromuscular Electrical Stimulation (“NM”), and Inferential (“IF”) therapy.

Portable electro-therapy devices provide a patient with the convenience of receiving therapy outside of a doctor's office or clinic. In particular, some portable electro-therapy devices utilize preset therapy protocols of either a single mode or mixed modes, from which a patient can select in order to perform stimulation therapy. Other portable devices can be programmed by a qualified technician, therapist or doctor with an appropriate therapy protocol.

With such devices, the prescribing doctor or therapist does not have information regarding how the device is being used by the patient when he or she is away from the doctor's office or clinic. In addition, the prescribing doctor or therapist may desire to change the therapy protocol for the patient without requiring the patient to bring the device in to be programmed with a new therapy protocol.

### SUMMARY OF THE INVENTION

The present invention solves the foregoing problem by providing a portable electro-therapy device which a doctor or therapist can remotely monitor, and which can be remotely programmed by the doctor or therapist with new therapy programs and protocols. The portable electro-therapy device interactively communicates with a server-based support system to provide usage and feedback information from the portable device, and to receive therapy protocols and/or programs, patient inquiries, and other information from the system. A doctor, therapist, technician or another authorized user can access the support system to see the usage, therapy history and data and feedback information uploaded from the portable device, and to update, modify and generate new therapy protocols and programs, as well as to generate inquiries for the patient and other information to be downloaded to the device.

According to one embodiment, a portable electro-therapy device of the present invention comprises a microprocessor configured to generate and control electrical stimulation output signals, an electrode jack, a plurality of electrodes operatively connected to the electrode jack and configured to transmit the electrical stimulation output signals to a patient, a modem communication port, and a modem configured to provide communication between the portable electro-therapy device and a server-based support system.

According to another embodiment of the present invention, a system for providing electrical stimulation therapy to a patient comprises a portable electro-therapy device having a modem, a microprocessor configured to generate and control output signals, an electrode jack and a plurality of electrodes operatively connected to the electrode jack and configured to transmit the output signals to the patient, and a server-based support system including an application server configured to provide authorized users with an interface to the server-based support system, a resources server configured to securely store patient data and a modem bank. The modem and the modem bank are configured to provide communication between the portable electrotherapy device and the server-based support system.

According to yet another embodiment of the present invention, a method for providing electrical stimulation therapy to a patient comprises the steps of downloading a therapy protocol from a server-based support system to a portable electro-therapy device, calculating, with a microprocessor of the portable electro-therapy device, electrical stimulation output signals based upon the downloaded therapy protocol, transmitting the electrical stimulation output signals to a patient through a plurality of electrodes connected to the portable electro-therapy device, uploading device data from the portable electro-therapy device to the server-based support system, and providing the device data to authorized users of the server-based support system through an application interface.

It is to be understood that both the foregoing summary of the invention and the following detailed description are exemplary and explanatory and are intended to provide further explanation of the invention as claimed.

### BRIEF DESCRIPTION OF THE DRAWINGS

The accompanying drawings, which are included to provide further understanding of the invention and are incorporated in and constitute a part of this specification, illustrate embodiments of the invention and together with the description serve to explain the principles of the invention. In the drawings:

FIG. 1 is a block diagram illustrating a portable electro-therapy device according to one embodiment of the present invention;

FIG. 2 illustrates a system for providing electrical stimulation therapy to a patient according to one embodiment of the present invention;

FIG. 3 is a flowchart illustrating the process by which a portable electro-therapy device periodically and automatically connects to a server-based support system, according to one embodiment of the present invention;

FIG. 4 is a flowchart illustrating the process executed by a server-based support system when portable electro-therapy device remotely connects, according to one embodiment of the present invention; and

FIG. 5 is a flowchart illustrating a method for providing electrical stimulation therapy to a patient according to one embodiment of the present invention.

### DETAILED DESCRIPTION OF THE INVENTION

In the following detailed description, numerous specific details are set forth to provide a full understanding of the present invention. It will be apparent, however, to one ordinarily skilled in the art that the present invention may be practiced without some of these specific details. In other instances, well-known structures and techniques have not been shown in detail to avoid unnecessarily obscuring the present invention.

According to one embodiment of the present invention, a portable electrical stimulator device provides electrical stimulation therapy to the patient. The portable electro-therapy device can interactively communicate, from a remote location, with a server-based support system to provide usage history, feedback, and other information to the support system, and to receive therapy protocols and/or programs, patient inquiries, and other information from the support system. A doctor, therapist, technician or other authorized user can access the server-based support system (e.g., through an application webpage) to view the usage and therapy history, feedback information and other data uploaded from the portable device, and to update, modify and generate new therapy protocols and programs, as well as to generate inquiries for the patient and other information to be downloaded to the device.

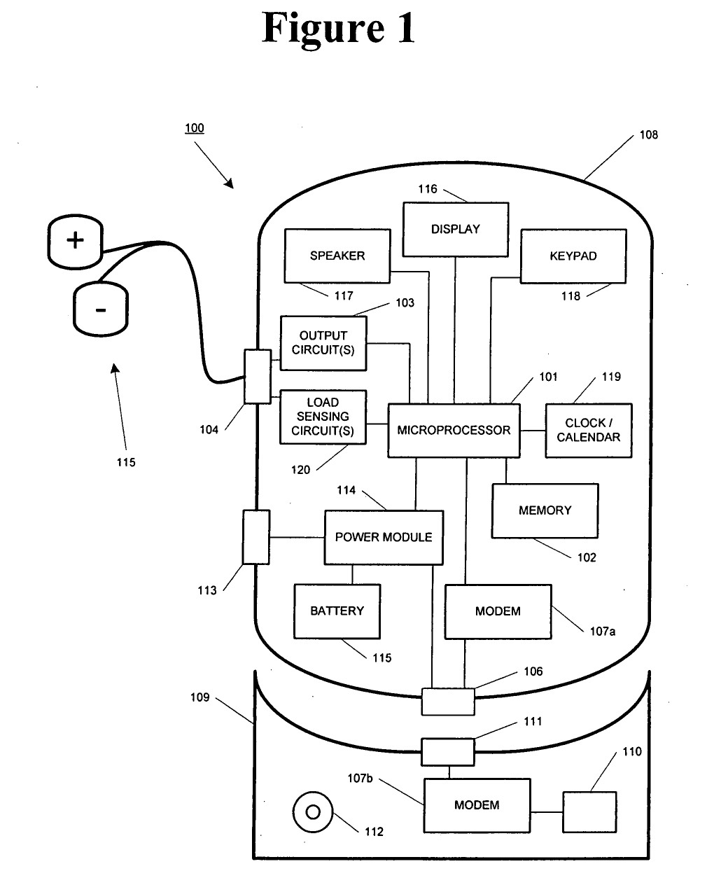

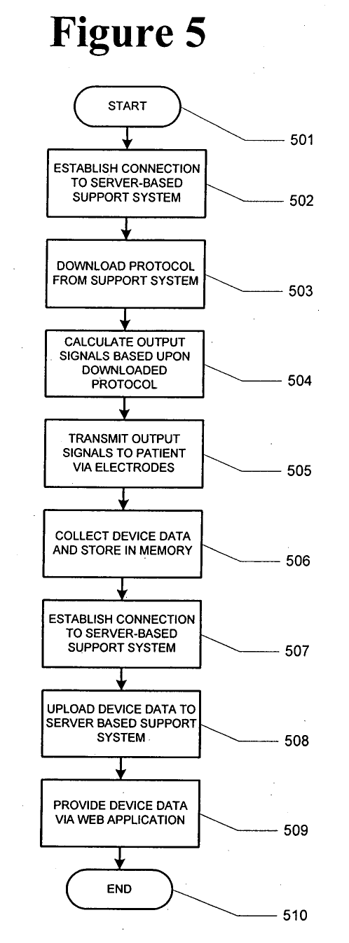

FIG. 1 illustrates a block diagram of the functional components of a portable electro-therapy device according to one embodiment of the present invention. Portable electro-therapy device 100 includes a processor such as microprocessor 101, memory 102, one or more output circuits 103 and an electrode jack 104. The terms “processor” or “microprocessor” are used herein to refer to any device or component thereof which is capable of executing code, calculating, or otherwise processing information, without limitation as to size, speed, configuration, and the like. The general operation of device 100 and the processing performed by device 100 to generate output signals in the various modes (IF, premodulated IF, N. Mex. and HVPC) are fully described in U.S. Pat. No. 6,564,103 B2, and the reader is referred thereto for the sake of brevity. The reader is particularly directed to FIGS. 5 to 23, and the related description, for the description of the operation of device 100, the formation and control of output stimulation signals, and the generation and control of output signals in the various modes, including IF, premodulated IF, N. Mex. and HVPC. In addition to the modes disclosed therein, device 100 may, according to various exemplary embodiments of the present invention, further provide additional modes including trans-cutaneous electrical nerve stimulation (“TENS”) and any other waveform known to those of skill in the art. In brief, according to one aspect of the present invention, information (e.g., a program or one or more sequences of instructions) is stored in memory 102 for providing electrical stimulation therapy to a patient. Execution of the program by microprocessor 101 causes microprocessor 101 to determine a mode of electro-therapy to provide, to calculate output signals based upon the determined mode of electrotherapy, and to transmit the output signals to the electrodes 115.

For example, according to various aspects of the present invention, microprocessor 101 may perform calculations based on information retrieved from memory 102 (e.g., a pre-programmed therapy mode or protocol), from user input through keypad 118 (e.g., user selected modes and adjustments, such as increasing or decreasing amplitude), from monitored output (e.g., from output circuits 103), or from feedback received from load sensing circuits 120 (e.g., if patient has disconnected the electrodes from his or her skin) to determine the signal to be output through output circuits 103 to the patient. The microprocessor 101 then outputs data to one or more appropriate output circuits 103 (e.g., one circuit for IF/NM output generation, another for HV output generation). The output circuit 103 forwards information to an output switching module in conjunction with timing signals from a timing controller, and the output switching module outputs the appropriate signal to the patient via electrodes 115 (e.g., reusable, self-adhering electrodes) through the proper corresponding connections in electrode jack 104.

According to one embodiment of the present invention, an output switching module allows the user of device 100 to define how two channels of stimulation output signals are assigned to the lead wires connected to electrode jack 104. In this regard, in an embodiment in which device 100 includes four electrodes 115, each with its own lead wire, the user, doctor or therapist is able to control how the output signals from two different channels of device 100 are assigned to the four lead wires. Device 100 provides this functionality through display 116 and keypad 118, using display screens to prompt the user (or doctor or therapist) to assign one channel's output signal to one pair of electrodes 115, and the other channel's output signal to another pair of electrodes 115. Similarly, according to another embodiment in which HVPC mode is utilized with five electrodes, the user can choose to use one, two or four lead wires as active lead wires, and the remainder as dispersive (non-active) wires.

According to another aspect of the present invention, device 100 also includes load sensing circuits 120 (e.g., an IF/NM output load sense module and a HV output load sense module), which are used to monitor the load of the output stimulation signals in each of the stimulation modes. The sensed load can be measured in either voltage or current, and can be used to determine whether there is an open circuit between the device and the patient, and can also be used to measure the resistance of the patient's tissue where electrodes 115 are connected. In IF and NM modes, current is controlled and monitored on the primary side of the transformer circuit for better control, and voltage to the patient is measured, thereby enabling the resistance load to be determined. In HVPC mode, the pulse width of the pulsed current is measured to determine the capacitor discharge time, from which resistance load can be determined. The determined resistance load is provided to microprocessor 101, which can determine based upon this information whether there is an open circuit in the lead wire and electrode connection, in which case display 116 may be used, together with an audible alarm through speakers 117, to warn the patient of the problem. Additionally, the resistance values determined by load sensing circuits 120 can be stored and later accessed by the user (or his therapist or doctor, through the remote server-based support system, discussed in greater detail below) to determine the health of the patient's tissue in the area of electrodes 115. Such information may be used to determine, for example, a level of tissue damage, nerve damage or cell damage in the area of electrodes 115. In this manner, load sensing circuits 120 act to control the appropriate output signals, to warn the user of a problematic connection of the lead wires and electrodes, and to provide resistance information of the patient's surrounding tissue.

According to one aspect of the present invention, as device 100 is being used, the output to the patient is monitored by microprocessor 101 and stored in memory 102 so that a physician (or other authorized individual) can view the stored information. The stored information may include data indicating, for example, when the device was used, for how long, together with any amplitude changes initiated by the user. By examining the stored output, the physician can evaluate the patient's usage of device 100 and the effect of the therapy on the patient in order to adjust, if necessary, the stored protocols. In this regard, according to one embodiment of the present invention, memory 102 is sufficiently large to permit device 100 to record approximately one hour's worth of usage activity in memory 102. After one hour, the oldest data is written over again in a continuously-looping fashion. In addition to diagnostic data, other data may be written over with continued usage as memory capacity limits are reached, according to an additional aspect of the present invention.

In accordance with another aspect of the present invention, device 100 also provides the functionality of a “SLEEP” mode, which can be set to start at a predetermined time and last for a predetermined duration, during which time no stimulation activity is provided. This may be useful in post-surgery settings in which the patient cannot operate device 100. In this regard, device 100 may be configured to operate in one or more scheduled stimulation modes, which are followed by a SLEEP mode, thereby allowing the patient to sleep for a predetermined time (i.e., without experiencing electrical stimulation). Alternatively, to conserve battery power, device 100 may be configured to automatically enter SLEEP mode after a predetermined amount of time has elapsed in which no stimulation activity or user-initiated activity occurs.

According to various aspects of the present invention, device 100 includes a number of additional features. When operating in neuromuscular mode, the output stimulation signal can operate in continuous mode with no (i.e., zero) off-time. Device 100 also includes a “placebo” mode, in which the user is led to believe that he or she is receiving electrical stimulation (regardless of the selected stimulation mode), but in which the clinical effect of any electrical stimulation is minimized (e.g., for placebo studies). In placebo mode, the actual amplitude of the output signal is determined without regard to the user's selected setting. According to one exemplary embodiment, the output stimulation signal in placebo mode is allowed to ramp from zero to only 80% of a selected amplitude setting over a period of three seconds, is held at 80% for one second, and is then reduced to zero again over the following two seconds. The output signal is held at zero for the next fifty-four seconds, and the cycle repeats again every minute. In this manner, the patient feels some electrical stimulation on a frequent basis, but may be generally unaware that the output signal is at zero level for the majority of the time.

According to yet another aspect of the present invention, device 100 may also have the capability to interject intra-pulse intervals into a pulsed waveform. In accordance with this aspect, a predetermined interval (delta-t) is interposed between the positive and negative components of each pulse in order to extend the transition between positive and negative components of the pulse. According to yet another aspect of the present invention, device 100 may also have a “vector” function, which is used in inferential mode to permit a user to adjust the point of intersection of two generated waveforms by using keypad 118 to shift the waveforms relative to each other.

According to yet another aspect of the present invention, device 100 allows the user to scroll though preset treatment protocols, to specify a particular order of modes to include in a single protocol, and to determine how many times (i.e., cycles) to execute the particular protocol. In addition, device 100 can save the last set of selected amplitudes used for one treatment protocol in memory 102. In this regard, when device is next used (or powered up) by the user, display 116 shows a screen asking the user if the last saved set of amplitudes should be used for the current session. In this manner, a commonly used amplitude setting can be carried from one therapy session to the next.

According to yet another aspect of the present invention, another feature of device 100 permits the output stimulation signal to be modified based on the size of electrodes used. This helps to avoid skin damage or discomfort to the patient. In the preset programming of device 100, the user can specify the electrode size. When a small electrode size is selected, the output stimulation signals may be reduced to 60% of the set intensity, and when normal electrode size is selected, the 100% level of the set intensity is permitted for the output stimulation signals.

Power can be provided to device 100 in any one of a number of ways, according to various embodiments of the present invention. Device 100 includes a battery 115 and a power module 114 for regulating power obtained from battery 115 and for charging battery 115 from an external power source. Battery 115 may include rechargeable lithium ion batteries, alkaline batteries or any other kind of batteries or fuel cells capable of providing power to device 100. Power module 114 can also condition the power received from an external source (such as an AC-to-DC power adaptor), monitor the power level drawn by device 100 and stored in battery 115, and reports the same to microprocessor 101. External power may be applied to device 100 through a first external power jack 113 in housing 108, or through a second external power jack 112 in cradle 109. Cradle 109 provides the power applied to second external power jack 112 to device 100 through communication connector 111.

According to various exemplary embodiments of the present invention, device 100 has a number of input/output functions for interacting with a patient. Device 100 includes a display 116 (optionally backlit) for providing visual information to a patient or other user, a speaker 117 (e.g., a buzzer, a loudspeaker, a beeper, or any other audible signaling device) for sounding alarms and providing other auditory information (e.g., alerting when an interface button is pushed at an inappropriate time) and a keypad 118 (optionally backlit) which includes a number of buttons for accepting input. Device 100 may also include clock and/or calendar circuits 119 for providing clock and/or calendar functionality and for maintaining and providing accurate time and date information for use by microprocessor 101. This functionality allows microprocessor 101 to determine when to use modem 107a or 107b to contact and communicate with a server-based support system, as is discussed in greater detail below.

According to various exemplary embodiments of the present invention, anyone using the device (e.g., a patient, a doctor, or a manufacturer) can turn the device on and off, select one of the stored modes, and adjust the amplitude (i.e., the strength) of the signal being output using keypad 118. Medical personnel (e.g., physicians) can be provided with an access code which permits access to additional functionality, such as the ability to define new modes and to modify stored modes. According to additional exemplary embodiments, the access code can be defined by a specific sequence of depressing the buttons of keypad 118, possibly including pressing multiple buttons simultaneously and/or pressing a button several times in succession and/or pressing one or more buttons for an extended period of time. According to an additional exemplary embodiment, the manufacturer may also have an access code which allows the manufacturer to perform calibration functions.

According to an additional aspect of the present invention, electrode jack 104 can also be used to support a connection to a computer or another external device in order to perform calibration or diagnostics on device 100, to download data stored in device 100 to a computer, or to upload firmware from a computer to device 100. In accordance with still other aspects of the invention, a calibration device may be connected device 100 via modem communication port 106 directly, or via cradle 109 when device 100 is set in cradle 109.

According to one embodiment of the present invention, device 100 is capable of communicating with a remote server-based support system, as described in greater detail below. Device 100 includes a modem communication port 106 and a modem 107a for providing communication between device 100 and the server-based support system. According to one embodiment of the present invention, communication port 106 may be an RS232 communication port capable of interfacing with a telephone connection, or for connecting directly to an external memory device, as is described in greater detail below. According to various aspects of the present invention, device 100 may include modem 107a within the housing 108 of device 100, or alternatively, may include a modem 107b within a cradle 109 with which device 100 can be operatively connected. Cradle 109 includes a telephone jack 110 (or network jack) for connecting to a network (e.g., a telephone network, LAN, etc.) and a communication connector 111 which can be coupled with modem communication port 106. Through communication connector 111 in cradle 109, device 100 can utilize telephone jack 110 to communicate with the remote server-based support system via the telephone network.

According to one aspect of the present invention, a program (e.g., one or more sequences of instructions) is stored in memory 102 for communicating with a server-based support system. Execution of the program by microprocessor 101 causes microprocessor 101 to determine if a predetermined time has elapsed, to establish a connection between device 100 and the server-based support system, to determine if there is device data to be uploaded from memory 102, to upload, if applicable, the device data from memory 102 to the server-based support system, to determine if there is server data to be downloaded from the server-based support system, and to download, if applicable, the server data from the server-based support system.

According to one embodiment of the present invention, device 100 can interactively communicate on a periodic basis, from a remote location, with a server-based support system. During this communication, device 100 can upload usage and feedback information from memory 102 of device 100 to the server-based support system, and can download therapy protocols and/or programs, patient inquiries, and other information to device 100 from the server-based support system. In this manner, a doctor, therapist, technician or other user can access the support system to view the usage, therapy history and data, and feedback information uploaded from portable device 100, and to update, modify and generate new therapy protocols and programs, as well as to generate inquiries for the patient and other information, which can then be downloaded to device 100 from the support system.

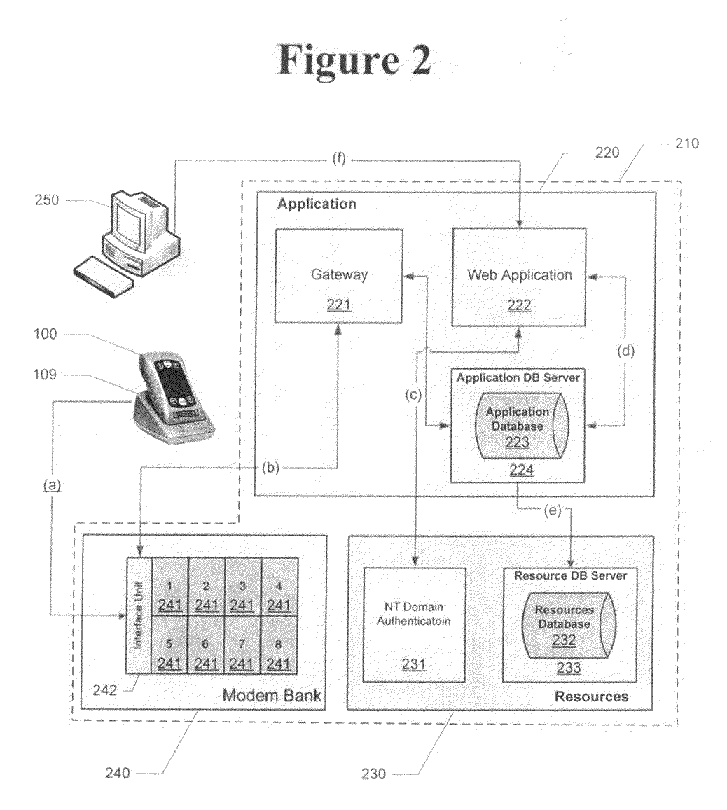

FIG. 2 illustrates a system for providing electrical stimulation therapy to a patient according to one embodiment of the invention. As seen in FIG. 2, the system includes both the portable electro-therapy device 100 and a server-based support system 210. Server-based support system 210 includes an application server 220, a resources server 230 and a modem bank 240. Application server 220 hosts several functional components, including at least gateway module 221, web application 222, application database 223 and application database server 224. Resources server 230 hosts several functional components, including at least NT domain authentication module 231, resources database 232 and resources database server 233. Modem bank 240 includes several modems 241 which, in this example, comprise eight modems, and interface unit 242 for interfacing both with telephone connections to various portable electro-therapy devices as well as with gateway module 221. The collection of modems operates in parallel to make connections to devices such as portable electro-therapy device 100 as they dial in. For example, with eight independent modems, the present exemplary embodiment is capable of accommodating eight simultaneous calls.

Application server 220 supports the communication and transfer of data between multiple portable electro-therapy devices and server-based support system 210. In addition, application server 220 supports the authorized access to server-based support system 210 by application users via one or more computers 250. In this regard, gateway module 221 interacts with device 100 via a modem 241 of modem bank 240. When a call comes in from device 100, gateway module 221 processes the call to (1) validate the integrity of the data, (2) look up any events that are waiting to occur (e.g., the application of new settings, etc.), (3) process usage data loaded by device 100 and (4) instruct device 100 to clear its internal storage. Application database 223 serves three main purposes: (1) it stores necessary data for interacting with device 100 (e.g., new presets), (2) it has a “linked server” connection to resources database 232 for retrieving patient and/or treatment information in real-time or near real-time and (3) it stores usage data uploaded by device 100 via modem bank 240. Web application 222 provides the web-based front end for the data warehouse of server-based support system 210. In the example shown in FIG. 2, web application 222 is utilized by authorized users (e.g., employees of the company that maintains server-based support system 210) as an Intranet application for accessing usage and feedback data from the many devices 100 that access server-based support system 210, and for performing related tasks such as running usage reports, setting presets on the device, etc. According to this exemplary embodiment, authorized users must log onto the company intranet domain and be a member of a specific security group to access and view web application 222. It should be appreciated that other embodiments of the invention allow web application 222 to be accessed by users outside the company that maintains server-based support system 210, such as by a WAN or the Internet, while providing appropriate security authorization to the outside users for such access.

Resources server 230 supports the secure maintenance of patient-related information and also the authentication of users allowed to access such information. In this regard, NT domain authentication module 231 controls access to resources server 230 and specifically to resources database 232. Resources database server 233 provides for communication with resources database 232. In the exemplary embodiment shown in FIG. 2, NT domain authentication module 231 is provided by Microsoft Active Directory via Windows Server. Web application 222 interrogates the NT domain authentication module 231 to ensure a putative user is actually a member of a specific NT Group before allowing this user to access any of the data in resources database 232. Resources database 232 functions as a “linked server” into web application 222, and thereby provides the benefit of storing all patient data in resources database 232, permitting web application 222 only to access this information under authorized conditions, but not to change any of the data contained therein. Accordingly, integrity and security of patient-related data is achieved.

In this regard, resources server 230 is preferably used in this exemplary embodiment to store patient data in a secure manner for those patients that use portable stimulation devices, and application server 220 is used to provide an interface to computer 250 for authorized users to access server-based support system 210 and to communicate with portable electro-therapy devices 100 via modem bank 240.

The interactions between the components shown in FIG. 2 are now described according to one embodiment of the present invention. When a device 100 dials in to the appropriate number (either directly through a modem communication port in device 100, or through cradle 109), a connection (a) (e.g., a telephone connection or other network connection) is established to the interface unit 242 of modem bank 240. Interface unit 242 receives the call from device 100, which is picked up by gateway module 221 via connection (b). Gateway module 221 uses serial communication ports to process the call from device 100 and manage the connection. During the call with device 100, data is transferred to and from device 100 and application database 232.

An authorized user (using computer 250) is authenticated by web application 222 by looking up the user's credentials in a directory, which in this example can be Microsoft's Active Directory. The user must be a member of a predetermined security group to interact with web application 222 via connection (f). If the user is not a member of one of these groups the request is rejected and access to web application 222 is denied. As discussed in greater detail above, the integrated security (connection (c)) between the web application 222 and resources database 232 is handled by NT domain authentication 231, which in this example uses Microsoft's Active Directory technology. When the user requests access to the web application 222 via an appropriate URL, NT domain authentication 231 ensures that the user is authorized to use web application 222 and access resources database 232. Once a session is established by a user with web application 222, a connection (d) is made to the application database 223 using a service account for retrieving the data. This service account can also be managed by a domain authentication service, such as Microsoft's Active Directory Technology to ensure credentials of the user are authorized and secure. As previously mentioned, servers 220 and 230 are linked (connection (e)) for authorized communication between application database 223 and resources database 232.

As is well-known to those of skill in the art, such linked database servers provide efficient access to remote data. This access allows web application 222 to store no patient data from resources database 232 other than relationships between a PatientID and a DeviceSN (serial number) in a cross reference table to support dialups and the storage of usage and feedback data. Like the web-to-database connection, this domain authentication is used to ensure credentials are secure before allowing the link between databases. This allows resources database 232 to maintain the security of secure data and to only provide essential data (by reference only) in web application 222. As previously indicated, nothing is written back from web application 222 to resources database 232.

While the exemplary embodiment described with reference to FIG. 2 has included a specific arrangement of servers and connections, it is expressly contemplated that the functionality of server-based support system 210 can be implemented with a single server, or in other combinations of servers and computing devices.

According to an additional aspect of the present invention, server-based support system 210 can also be used to upload updated firmware or new firmware to device 100. Also, in accordance with further aspects of the present invention, server-based support system 210 can also be used to set other preset data and other function parameters in device 100.

According to yet another aspect of the present invention, an external memory device, such as a data key (e.g., memory stick), can be connected to modem communication port 106 of device 100 to download usage data and feedback information from device 100 to the external memory device. The external memory device can then be sent (e.g., via the postal service or other parcel carrier) to a service center where it can be connected to server-based support system 210 via gateway module 221. For this reason, gateway module 221 may be equipped with a standard communication port (e.g., USB, serial, RS-232, etc.) which can accommodate external memory devices such as memory sticks, keys, and any other external memory devices. In this manner, the usage data and feedback information can be uploaded from the external memory device to server-based support system 210. In addition, server-based support system 210 can download new events, therapy protocols and programs, and inquiries to the external memory device which can then be returned to the user (e.g., patient, doctor, therapist, etc.) and connected to device 100, whereupon the new events, therapy protocols and programs, and inquiries can be uploaded to device 100.

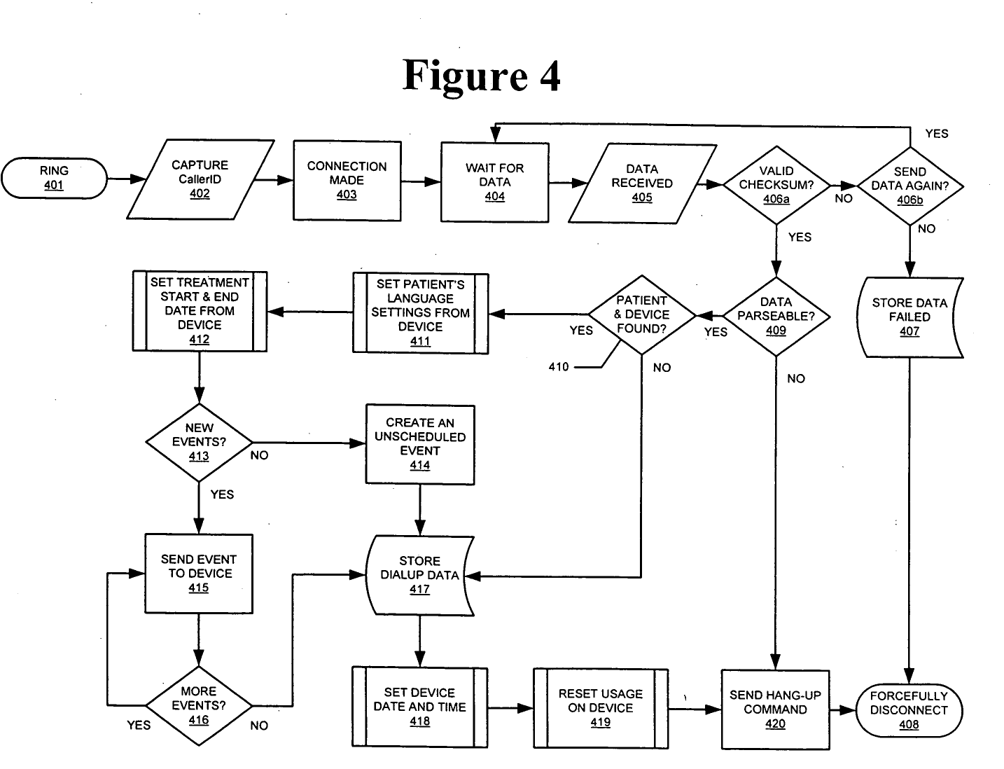

Alternatively, in an embodiment in which cradle 109 includes a modem 107b, the external memory device may be operatively connected to a port on cradle 109 (which acts as a data transceiver device). In this embodiment, cradle 109 can transmit the usage data and feedback information through interface unit 242 to the server-based support system, as described in greater detail below with respect to FIG. 4. Accordingly, the present invention has application to server-based support systems which communicate with a number of different therapy devices. Such devices may either communicate with the server-based support system by docking in a cradle, as described in greater detail above, or by transferring patient data and therapy information to an intermediate medium, such as the electronic memory key described above, which is in turn docked with a cradle or data transceiver device. According to various embodiments of the present invention, said cradles or data transceiver devices may include any one of a number of different kinds of modems (e.g., a telephone modem, a wired network card, a wireless network card, a wireless carrier card, etc.).

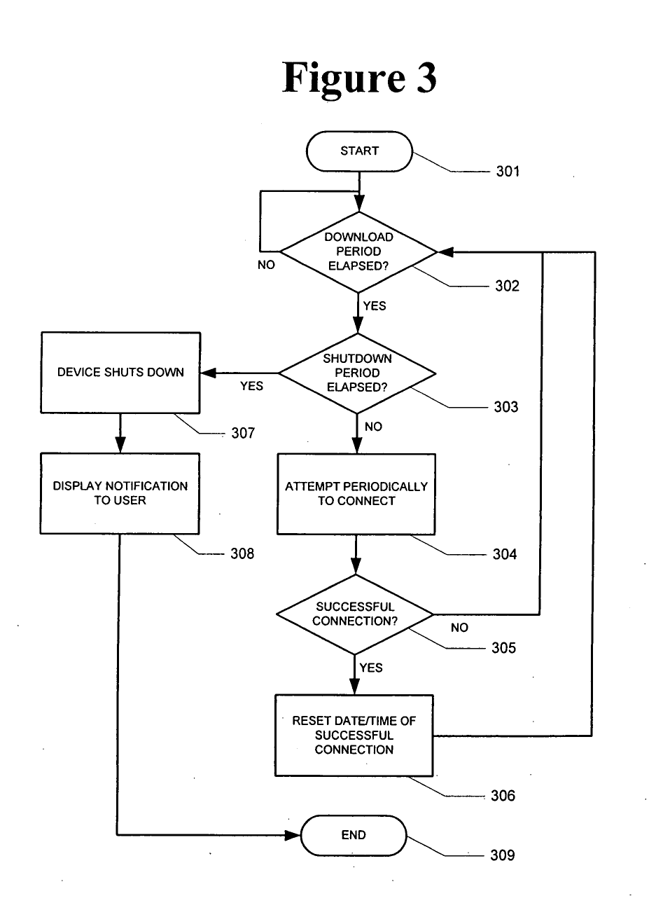

FIG. 3 is a flowchart to explain an example of the logic engaged by microprocessor 101 of device 100 to automatically connect to server-based support system 210 on a periodic basis, according to one exemplary aspect of the invention. In order for device 100 to make these automatic connections, the user/patient of device 100 docks device 100 into cradle 109 during a specified time period when it will not be used for therapy, such as at night time. In this manner, device 100 can recharge battery 115 and also automatically connect to server-based support system 210 while docked in cradle 109. As seen in FIG. 3, the process starts at step 301 when the user/patient first obtains device 100. In step 302, it is determined whether a predefined “download time-period” has expired since the last successful connection (saved in memory 102) by utilizing the date and time from clock 119. For example, the predefined “download time-period” can be set to seven days so that device 100 will attempt a connection with server-based support system 210 seven days after the last successful connection. It should be appreciated that the predefined “download time-period” can be set/modified by a user of device 100 or through server-based support system 210. If it is determined in step 302 that a predefined “download time-period” has expired since the last successful connection, then process flow passes to step 303. Otherwise, process flow simply loops back to step 302 to continually check (at intervals) whether the “download time-period” has expired.

In step 303, it is determined whether a predefined “shut-down” has expired since the last successful connection. In one example, the “shut-down” time period is set by the manufacturer/distributor of device 100 to shut down the device if a significant amount of time has gone by since device 100 last successfully connected to server-based support system 210. For example, the “shut-down” time period could be set to ninety days by the therapist, doctor, or manufacturer/distributor of device 100, so that if ninety days passes without a successful connection from device 100 to server-based support system 210, then device 100 will be shut down so the user can no longer use it for therapy. This has the benefit of preventing device 100 from being used by someone other than the authorized patient/user, and of preventing the patient/user from continuing to use a treatment protocol that may no longer be appropriate for that patient. Accordingly, in exemplary aspects of the invention, the “shut-down” time period is not a parameter that can be set or modified by the patient/user of device 100.

If, in step 303, it is determined that the predefined “shut-down” has expired since the last successful connection, then process flow passes to step 307 in which the device shuts down and is no longer operational for purposes of outputting stimulation signals for therapy treatment. Flow then passes to step 308 in which a display screen is shown in display 200 to inform the user that the device is no longer operational and to contact an appropriate person, such as the therapist, doctor or technician, and flow passes to “end” in step 309. If in step 303, it is determined that the predefined “shut-down” has not expired since the last successful connection, then process flow passes to step 304 in which device 100 attempts periodically to connect to server-based support system 210 using modem 107a or 107b, but only during a specified time range. For example, device 100 can be set to attempt to dial-in through modem 107a or 107b every 15 minutes until a successful connection is established, but only during the hours of 2:00 a.m. to 4:00 a.m., which is a likely time when device 100 will be docked in cradle 109. The re-try frequency and the allowed time range can be set by the user (or by the therapist, doctor or technician) either manually or through server-based support system 210. After the allowed time range has expired (e.g., after 4:00 a.m.), flow passes to step 305 in which it is determined if a successful connection was established between device 100 and server-based support system 210 in step 304. If it is determined in step 305 that a successful connection was established in step 304, then process flow passes to step 306, in which the saved date and time of the last successful connection is updated. Flow then passes from step 306 to step 302, in which the process starts over to continue monitoring for the next time that a connection should be attempted, based on the updated date/time of the recent successful connection.

In this manner, device 100 automatically attempts to dial-in to server-based support system 210 without the need for intervention by the user/patient. Device 100 can therefore periodically download usage and feedback data, as well as other device data, to server-based support system 210 for subsequent monitoring/review by an appropriate doctor, therapist, or technician, as the case may be. In addition, server-based support system 216 can upload new firmware, device settings, treatment protocols, patient inquiries, and other data to device 100 on a periodic basis when device 100 dials-in to server-based support system 210.

While the foregoing exemplary embodiment has been described with respect to device 100 being docked in cradle 109, it will be readily apparent to those of skill in the art that the scope of the present invention is not limited to such an arrangement. Rather, according to an additional embodiment of the present invention, device 100 may connect to a telephone or other network through modem communication port 106 directly, without being docked in cradle 109, as device 100 may include a modem 107a within its housing 108.

FIG. 4 is a flowchart to explain the process executed by server-based support system 210 when device 100 (or cradle 109, when cradle 109 is acting as a data transceiver device) dials-in for a connection, particularly in gateway module 300. As seen in FIG. 4, the process starts at step 401 in which the “ring” is received from device 100 at interface unit 242 of modem bank 340. In step 402, the caller ID is captured and a connection (handshake) is made in step 403 between device 100 and gateway module 300 via modem 241. Next, gateway module 300 awaits data to be transferred from device 100 via modem 241, and the data is received in step 405. In exemplary aspects of the invention, the data is transferred in predefined blocks between device 100 and server-based support system 210. The checksum is checked for validity in step 406a and, if valid, flow passes to step 409. If the checksum is not valid, then there was an error in the transmission of data from device 100 to gateway module 300, and the flow passes to step 406b, in which it is determined whether the data should be resent, based upon how many attempts have already been made. If the pre-determined number of attempts has not been made, flow returns to step 404. If the pre-determined number of attempts has been unsuccessfully made, flow passes to step 407 in which the failed data received is stored in application database 223. In such a case, flow then passes to step 408 in which the connection is terminated.

If the checksum was valid, then it is determined if the received data can be parsed in step 409. If the data cannot be parsed, then flow passes to step 420 in which a hang-up command is sent to modem 241 and then the connection is terminated in step 408. Otherwise, if the data can be parsed, flow passes to step 410 in which the data is parsed for the PatientID and the DeviceSN. If these two pieces of data are found, then flow passes to step 411. Otherwise, flow passes to step 417 in which the dial-up data is stored in application database 223, and then the device date/time data is set through the modem connection in step 418, and the recent usage data block of the device is reset (erased) in step 419, after which flow passes to step 420 in which the hang-up command is issued and the connection is terminated in step 408.

If, in step 410, the PatientID and the DeviceSN are found in the received data, then flow passes to step 411 in which the patient's language setting is obtained from the device and is set in application database 223, in correspondence with the PatientID and DeviceSN. In such case, flow then passes to step 412 in which the treatment start and end date is obtained from the device and is set in application database 223, in correspondence with the PatientID and DeviceSN. Next, it is determined if new events have been set-up by web application 222 for this patient/device in step 413 and, if so, flow passes to steps 415 and 416 in which all events for this patient/device are uploaded to device 100. As discussed above, the events to be uploaded to device 100 can include new treatment protocols, device settings, firmware and inquiries to the patient to obtain feedback from the patient (regarding pain level, equipment reorder, etc.). Then flow passes to steps 417 through 420 (discussed above) and on to termination of the connection in step 408.

According to one aspect of the present invention, web application 222 hosted in server-based support system 210 provides accessibility to and interaction with server-based support system 210 according to a method which employs the steps of: (1) requesting and validating authorization data from the user, (2) presenting an application home page to the user through which the user can view, monitor and search for device usage data and feedback data from at least one portable electro-therapy device, (3) presenting, at the user's request, an event settings page through which the user can set-up modified or new therapy protocols and settings to be downloaded to a particular portable electro-therapy device, (4) presenting, at the user's request, an event questions page through which the user can set-up modified or new questions for the patient to be downloaded to a particular portable electro-therapy device, (5) presenting, at the user's request, a report generation page through which the user receives generated reports based on usage and feedback data and device settings retrieved from one or more portable electro-therapy devices, and (6) presenting, at the user's request, an administrative page through which the user can view system usage and settings, such as a gateway log to view access to the system through the gateway module by various portable electro-therapy devices and the data transferred from such devices.

As discussed above in greater detail, authorized users can access web application 222 through computer(s) 250 upon proper authentication, according to one aspect of the present invention. Web application 222 then presents various web pages to the authenticated user to access device usage and patient feedback information that was downloaded from device(s) 100. In this regard, different users can have different levels of access allowed and can therefore access only certain sets of device/patient data and certain functions. Of course, some users, such as administrators, can access all device/patient data and all functions through web application 222.

According to one aspect of the present invention, a home page is provided which allows an authorized user to search (according to allowed access) the application database 223 for data downloaded from devices. The user can search according to various parameters such as PatientID, DeviceSN, the user's ID, and other parameters. For example, a doctor or therapist can search for recent usage data for all devices belonging to patients of that doctor/therapist.

According to additional aspects of the present invention, a user of web application 222 can set up a treatment protocol as a preset to be downloaded to device 100. For example, the user of web application 222 can select from various predefined Master Treatments and also from Master Presets within each Master Treatment class. The new treatment protocol event is scheduled to be applied to device 100 starting on a specific date. The user of web application 222 can also enter custom question (in whatever language the patient speaks), and can specify the Answer Type (e.g., True/False, 1-10, etc.) and the Question Frequency (e.g., before treatment, after treatment, once per day, etc.). In this manner, a therapist, doctor or technician can remotely obtain feedback information from the patient regarding their treatment with device 100.

According to an additional aspect of the present invention, the web application and the electro-therapy device can be configured to operate (e.g., both outputting and accepting input) in any language. For example, for a patient with limited English-language skills, the device can be configured to display information and prompt for patient input using the patient's native language. Similarly, the web application interface (and information about a patient) may be provided in any language, including without limitation English, Spanish, Vietnamese, etc.

It should be appreciated that many other functions can be supported by web application 222. In this regard, other functions supported in exemplary embodiments include generating reports based on device usage history and answers to custom questions, administrative tools such as real-time display of the gateway log to show dial-in activity from devices to modem bank 240, and administrative data checking to allow an authorized user of web application 222 to review the actual data received from devices that have dialed-in to gateway module 221 via modem bank 240.

Turning to FIG. 5, a flowchart illustrating a method for providing electrical stimulation therapy to a patient is provided. After the process begins in step 501, a connection is established in step 502 between a portable electro-therapy device, such as device 100, and a server-based support system, such as system 210. In step 503, a therapy protocol is downloaded from the server-based support system to the portable electro-therapy device. A therapy protocol may be configured to provide output signals to a patient in one or more therapy modes for various durations and at various strengths. In step 504, a microprocessor of the portable electro-therapy device calculates one or more output signals to be generated, based upon the downloaded therapy protocol. In step 505, the output signals are transmitted to a patient through the electrodes connecting the patient to portable electro-therapy device. During the operation of the portable electro-therapy device, device data (e.g., device usage history, settings, circuit loads, patient input, etc.) is collected and stored in the device's memory. In step 507, a connection is again established between the portable electro-therapy device and the server-based support system. In step 508, the device data that was collected in step 506 is uploaded from the memory of the portable electro-therapy device to the server-based support system. During this step, additional data (e.g., another therapy protocol, firmware updates, questions for the patient, etc.) may be downloaded to the portable electro-therapy device. In step 509, the device data that was uploaded in step 508 is provided to authorized users of the server-based support system through an application interface, such as web application 222.

While the present invention has been particularly described with reference to the various figures and embodiments, it should be understood that these are for illustration purposes only and should not be taken as limiting the scope of the invention. There may be many other ways to implement the invention. Many changes and modifications may be made to the invention, by one having ordinary skill in the art, without departing from the spirit and scope of the invention.

What is claimed is:

1. A portable electro-therapy device comprising:
a microprocessor configured to generate and control electrical stimulation output signals;
an electrode jack;
a plurality of electrodes operatively connected to the electrode jack and configured to transmit the electrical stimulation output signals to a patient;
a modem communication port; and
a modem configured to provide communication between the portable electro-therapy device and a server-based support system.

2. The portable electrotherapy device of claim 1, further comprising a cradle having a communication connector configured to be operatively coupled with the modem communication port.

3. The portable electro-therapy device of claim 2, wherein the modem of the portable electro-therapy device is located within the cradle.

4. The portable electro-therapy device of claim 2, wherein the communication connector is configured to provide power to the portable electro-therapy device.

5. The portable electro-therapy device of claim 1, wherein the modem communication port and the electrode jack comprise a single interface.

6. The portable electro-therapy device of claim 1, further comprising a memory configured to store one or more of: therapy protocols, therapy programs, device data, usage data and feedback data.

7. The portable electro-therapy device of claim 6, wherein the memory includes one or more sequences of instructions for providing electrical stimulation therapy to the patient, wherein the execution of the one or more sequences of instructions by the microprocessor causes the microprocessor to perform the steps of:
determining a mode of electro-therapy to provide;
calculating the electrical stimulation output signals based upon the determined mode of electro-therapy; and
transmitting the electrical stimulation output signals to one or more of the plurality of electrodes.

8. The portable electro-therapy device of claim 6, wherein the memory includes one or more sequences of instructions for communicating with the server-based support system, wherein the execution of the one or more sequences of instructions by the microprocessor causes the microprocessor to perform the steps of:
determining if a predetermined time has elapsed;
establishing a connection between the portable electro-therapy device and the server-based support system;
determining if there is device data to be uploaded from the memory;
uploading, if there is device data to be uploaded, device data from the memory to the server-based support system;
determining if there is server data to be downloaded from the server-based support system; and
downloading, if there is server data to be downloaded, server data from the server-based support system.

9. The portable electro-therapy device of claim 1, further comprising a load-sensing circuit configured to sense an output load to the plurality of electrodes.

10. The portable electro-therapy device of claim 1, wherein one of the electrode jack and the modem communication port is configured to operatively connect with an external device for communicating data to and from the external device.

11. The portable electro-therapy device of claim 10, wherein the external device is selected from the group consisting of: a computer, an external memory device and a calibration device.

12. The portable electro-therapy device of claim 1, further comprising a battery configured to provide power to the portable electro-therapy device and a batter charger configured to charge the battery from an external power source.

13. The portable electro-therapy device of claim 1, further comprising at least one of: a display, one or more interface buttons, a speaker, a clock module and a calendar module.

14. The portable electro-therapy device of claim 1, wherein the electrical stimulation output signals are selected from the group consisting of interferential output signals, pre-modulated interferential output signals, neuromuscular output signals, high volt pulse current output signals, trans-cutaneous electrical nerve stimulation and combinations thereof.

15. The portable electro-therapy device of claim 1, wherein an intensity of the electrical stimulation output signals is scalable to accommodate electrodes of differing sizes.

16. A system for providing electrical stimulation therapy to a patient, comprising:
a portable electro-therapy device having a modem, a microprocessor configured to generate and control output signals, an electrode jack and a plurality of electrodes operatively connected to the electrode jack and configured to transmit the output signals to the patient; and
a server-based support system including an application server configured to provide authorized users with an interface to the server-based support system, a resources server configured to securely store patient data and a modem bank,
wherein the modem and the modem bank are configured to provide communication between the portable electro-therapy device and the server-based support system.

17. The system of claim 16, wherein the application server and the resources server comprise a single server.

18. The system of claim 16, wherein the resources server includes a patient information database.

19. The system of claim 16, wherein the application server includes one or more of: a gateway module for interfacing with the modem bank, a web application module for allowing authorized users to access the server-based support system, and a device information database.

20. The system of claim 16, wherein the communication between the portable electro-therapy device and the server-based support system communicates one or more of: usage data and feedback information uploaded from the portable electro-therapy device.

21. The system of claim 16, wherein the communication between the portable electro-therapy device and the server-based support system communicates one or more of: therapy protocols, programs and inquiries downloaded from the server-based support system.

22. The system of claim 16, wherein a memory of the portable electro-therapy device includes one or more sequences of instructions for communicating with the server-based support system, wherein the execution of the one or more sequences of instructions by the microprocessor causes the microprocessor to perform the steps of:
determining if a predetermined time has elapsed;
establishing a connection between the portable electro-therapy device and the server-based support system;
determining if there is device data to be uploaded from the memory;
uploading, if there is device data to be uploaded, device data from the memory to the server-based support system;
determining if there is server data to be downloaded from the server-based support system; and
downloading, if there is server data to be downloaded, server data from the server-based support system.

23. A method for providing electrical stimulation therapy to a patient, comprising the steps of:
downloading a therapy protocol from a server-based support system to a portable electro-therapy device;
calculating, with a microprocessor of the portable electro-therapy device, electrical stimulation output signals based upon the downloaded therapy protocol;
transmitting the electrical stimulation output signals to a patient through a plurality of electrodes connected to the portable electro-therapy device;
uploading device data from the portable electro-therapy device to the server-based support system; and
providing the device data to authorized users of the server-based support system through an application interface.

24. A system for maintaining therapy information about a patient, comprising:
a server-based support system including an application server configured to provide authorized users with an interface to the server-based support system and a resources server configured to securely store patient data and a gateway module; and
a data transceiver device operatively connected to the server-based support system and configured to receive the patient data from an external memory and to transmit the patient data to the gateway module.

25. The system according to claim 24, wherein the application server and the resources server comprise a single server.
